(19) United States
(12) Patent Application Publication
(10) Pub. No.: US 2026/0004902 A1
(43) Pub. Date: Jan. 1, 2026

(54) AUTOMATED ALERT SYSTEM

(71) Applicant: Joint Technology Solutions, Inc., Manassas, VA (US)

(72) Inventors: Santiago Valdizan, Fairfax, VA (US); Tania Garavito, Haymarket, VA (US)

(73) Assignee: Joint Technology Solutions, Inc., Manassas, VA (US)

(21) Appl. No.: 19/320,613

(22) Filed: Sep. 5, 2025

Related U.S. Application Data
(63) Continuation of application No. 15/867,810, filed on Jan. 11, 2018, now abandoned.
(60) Provisional application No. 62/501,275, filed on May 4, 2017.

(51) Int. Cl. G16H 10/60; G16H 40/20; G16H 50/20

(52) U.S. Cl. CPC G16H 10/60; G16H 40/20; G16H 50/20

(57) ABSTRACT

Automated alert system software may interface with an electronic medical records (EMR) system and provide interested individuals with immediate notifications of critical changes during a patient's course of treatment in a hospital. This may reduce or minimize the amount of time it takes to notify families of critical moments during a hospital admission, for example. The automated alert system software may interface with the hospital EMR system by directly reading and extracting the necessary information from the database, such as the patient name and emergency contact information.

### CROSS REFERENCE TO RELATED APPLICATIONS

The present application is a continuation of and claims the benefit of co-pending U.S. Non-Provisional application Ser. No. 15/867,810, filed Jan. 11, 2018, which claims the benefit of U.S. Provisional App. No. 62/501,275, filed May 4, 2017, each of which is hereby incorporated by reference in its entirety as if fully set forth herein.

### FIELD

The present invention generally relates to an automated alert system, and more specifically, to an automated alert system that interfaces with an electronic medical records system to provide automated notifications to authorized individuals regarding changes that occur during a patient's medical treatment.

### BACKGROUND

During a hospital admission/course, communication is often needed between healthcare professionals and family members as it relates to medication reconciliation, medical history, change in location, and changes in health status (i.e. code blue, decompensation). Current hospital Standard Operating Procedures (SOP) do not offer an effective family notification procedure for changes in a patient's medical status, such as a patient code blue (i.e., delays due to all medical personnel tending to patient and inability to notify family of changes in medical status). During these times, the hospital provides medical solutions while the patient is in a code blue that are not aligned with the patient's desires or the intent of the family. This allows unnecessary or unwanted hospital procedures to be performed, creating an additional financial burden (e.g., oversight on unnecessary medical procedures) and creating potential risks to the hospital industry. Furthermore, when changes in medical status do occur, family members are not properly contacted (i.e., wrong phone numbers are listed, no immediate notification is provided, etc.). As a result of these issues, for instance, family members are not able to provide proper comfort to their loved ones when critically ill or passing.

Conventional notification processes are not effective for both family members and treatment teams since the only way to notify families during a change in medical status was later in the treatment process. This delay in notification causes unnecessary grief to the family for not being closer to their loved one and allows unnecessary hospital procedures to occur as a result of the delay in notification. Accordingly, an improved notification process and system may be beneficial.

### SUMMARY

Certain embodiments of the present invention may provide solutions to the problems and needs in the art that have not yet been fully identified, appreciated, or solved by conventional automated alert systems. For example, some embodiments pertain to an automated alert system that interfaces with an electronic medical records system to provide automated notifications to authorized individuals (e.g., family, friends, guardians, etc.) regarding changes that occur during a patient's medical treatment. Such embodiments may reduce or minimize the amount of time it takes to notify interested individuals of critical events during a hospital admission. A benefit of this software to healthcare providers in some embodiments, which may at least in part be in the form of a cloud-based package, is that it may effectively focus the efforts of treatment teams, allowing them to adequately and correctly apply the correct course of action as it relates to the current patient status. This software may interface with a hospital emergency medical records (EMR) system by directly reading and extracting the necessary information from the database, such as the patient name and emergency contact information. This information may be necessary to ensure proper synchronization of the patient record with the software of some embodiments, closing a critical gap within the healthcare industry by addressing risks areas including, but not limited to, communication of direct and indirect benefits regarding inpatient and outpatient costs. These and other benefits may positively impact healthcare insurance costs as well.

In an embodiment, a system includes a medical professional workstation configured to run a hospital EMR application and an EMR message monitor application. The system also includes an application server configured to run a server-side automated alert application that operates in concert with the EMR message monitor application. The system further includes a user device configured to run a user automated alert application that operates in concert with the server-side automated alert application. The EMR message monitor application is configured to interface with the hospital EMR application and monitor for one or more changes a condition of a patient that require notification of an authorized individual associated with the user device.

In another embodiment, a computer-implemented method includes monitoring, by an EMR message monitor application, for one or more changes a condition of a patient that require notification of an authorized individual associated with a user device. When one or more changes in the patient's condition are detected that require notification of the authorized individual associated with the user device, the computer-implemented method also includes executing a notification procedure, by the EMR message monitor application, by transmitting a message including information pertaining to the one or more changes in the condition of the patient to a server-side automated alert application.

In yet another embodiment, a computer-implemented method includes detecting a patient condition change in an EMR application, by an EMR message monitor application. The computer-implemented method also includes sending a message to a server-side automated alert application running on an application server that includes information about detected patient condition change, by the EMR message monitor application.

### BRIEF DESCRIPTION OF THE DRAWINGS

In order that the advantages of certain embodiments of the invention will be readily understood, a more particular description of the invention briefly described above will be rendered by reference to specific embodiments that are illustrated in the appended drawings. While it should be understood that these drawings depict only typical embodiments of the invention and are not therefore to be considered to be limiting of its scope, the invention will be described and explained with additional specificity and detail through the use of the accompanying drawings, in which:

FIG. 1 is an architectural diagram illustrating an automated alert system, according to an embodiment of the present invention.

FIG. 2 is a flowchart illustrating a process for an automated alert system, according to an embodiment of the present invention.

FIG. 3 is a flowchart illustrating a process for an automated alert system, according to an embodiment of the present invention.

FIG. 4 is a block diagram illustrating a computing system configured to provide an automated alert system, according to an embodiment of the present invention.

### DETAILED DESCRIPTION

Some embodiments of the present invention pertain to an automated alert system that interfaces with an electronic medical records system to provide automated notifications to authorized individuals (e.g., family, friends, guardians, etc.) regarding changes that occur during a patient's medical treatment. The automated alert system of some embodiments notifies family members and/or other authorized individuals of critical health care changes of their loved ones. Depending on the type of changes recorded in emergency medical records (EMRs) for a patient, the automated alert system in some embodiments automatically establishes a communication bridge between interested individuals and hospital staff members (e.g., doctors, nurses, etc.). This ensures a healthy collaboration environment that serves the patient's wishes.

As stated above, there is currently an unsolved problem in the healthcare industry as it lacks notification procedures between hospital staff and interested individuals. During a patient's hospital admission and course of treatment, communication is often needed between healthcare professionals and family members regarding medication reconciliation, medical history, changes in location, and changes in health status (e.g., code blue, decompensation, etc.). Current hospital Standard Operating Procedures (SOPs) do not offer an effective family notification procedure with respect to changes in a patient's medical status, such as a patient code blue. During a patient code blue, for example, the hospital may provide medical treatment that is not aligned with the patient's desires or the family's intent. This unwanted or nonbeneficial medical treatment may create an additional financial burden during hospital admission, and also create potential risks for the hospital industry. Furthermore, when changes in medical status do occur, family members are not properly contacted (i.e., wrong phone numbers may be listed, no immediate notification is provided, etc.). The automated alert system of some embodiments may address this and other problems, filling a critical gap in the health care industry.

Conventionally, hospitals designate a person from the treatment team during a change in medical status to attempt to reach family members. This wastes critical time and resources of the designated person, which would be better spent treating patients. Furthermore, designating an individual from the treatment team invites errors. Accordingly, some embodiments provide automated notifications that rapidly reach interested individuals as the treatment team members attempt to stabilize and treat the patient.

When relatives or other designated personnel should be notified regarding a critical condition of the patient, authorized medical personnel may access an EMR message monitor application through any suitable EMR input terminal (e.g., a nurse desktop workstation) to send a message to indicate that a critical medical event has occurred. This request should be made when a callback number can be provided that is known to be answered by someone authorized to provide detailed information about the event triggering the call. At the discretion of the authorized hospital personnel, any suitable time-sensitive message (e.g., text, email, voicemail. Etc.) can be sent using the automated alert system. In some embodiments, this functionality may be applied to an outpatient care or nursing home setting. This software solution provides an improvement to current standards of care by creating a simple and effective family notification procedure.

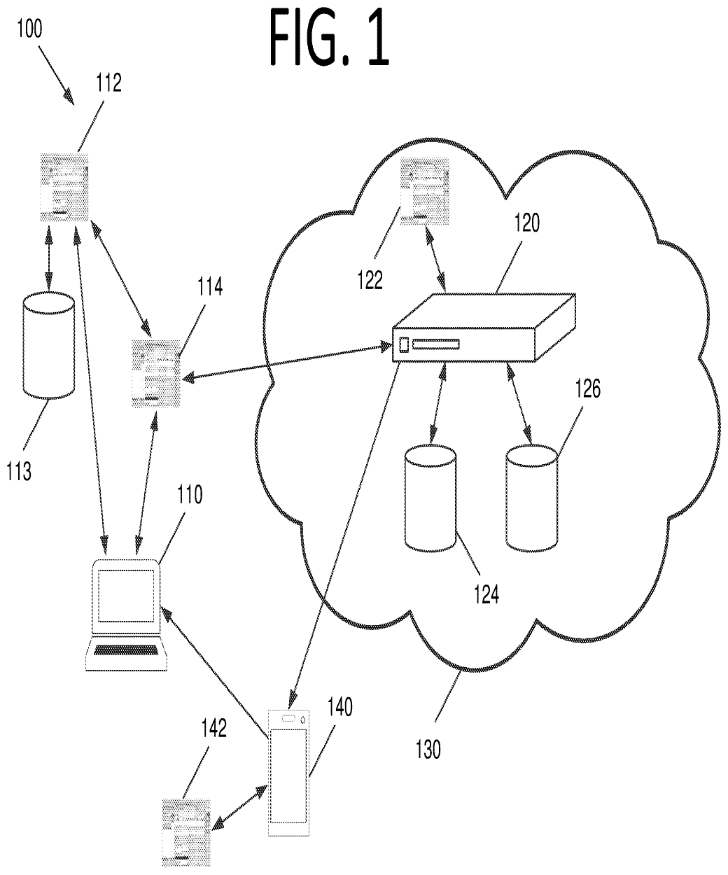

The automated alert system of some embodiments provides an automated notification system between medical personnel and authorized individuals. Such an automated alert system 100 is shown in FIG. 1. Automated alert system 100 includes a nurse workstation 110 (or any other suitable medical professional workstation), a hospital EMR application 112 with access to a personal health records (PHR) database 113, an EMR message monitor application 114, an application server 120 configured to run a server-side automated alert application 122, and a user device 140 configured to run a user automated alert application 142. While shown as a cell phone here, user device 140 could be a laptop, a desktop computer, a tablet, a smart watch, or any other suitable user device without deviating from the scope of the invention.

Hospital EMR application 112 may be a standard health care tool where practitioners, doctors, nurses, and/or faculty staff can access patient data or execute critical updates on system information that modifies a patient record. Hospital EMR application 112 runs on nurse workstation 110 in this embodiment and is implemented in software. EMR message monitor application 114, which also runs on nurse workstation 110, interfaces with EMR application 112 and searches for a series of conditions that may be critical and necessary to activate and notify interested individuals of a patient's status. For instance, in some embodiments, EMR message monitor application 114 may read a patient's recorded temperature information, seeking critical values for key algorithms.

These key algorithms may read the Unique Resource Identifier (URI) component associated with the resource being monitored by EMR message monitor application 114 and perform a ternary check on the resources being updated, checking the instance's meta attribute. If this meta attribute has changed and there is an EPOCH change, for instance, the domain resource's “valueQuanity” attribute and coding value may be determined. In some embodiments, the valueQuantity is attributed by the “Observation” resource. The observation's “patient's recorded temperature information” may be defined as URI values assigned to observational LOINC® codes (see https://loinc.org). A patient's temperature may be in the Observational subcategory vital-signs, which may be nomenclated as universal unique identifiers. A patient's body temperature has a coding value of 8310-5 and the patient's oral temperature has a coding value of 8333-1 under the LOINC standard.

EMR message monitor application 114 accepts an MXN spreadsheet (cardinality of 5 . . . , 1 . . . respectively) and iterates through each row in this embodiment. The header for each cell in a row corresponds to the conditions “Resource,” “Type,” “Operation,” “Value,” or “Resolution.” If resource, type, operation, and value match the instance's characteristics and the condition is evaluated as true, some embodiments immediately trigger and perform an appropriate API call, incumbent on the Resolution defined for this condition case expressed herein.

Department records and credentials are stored in PHR database 113, which may be implemented in software on nurse workstation 110 or stored on another computing system accessible by nurse workstation 110. When such critical information been located and processed, identifying a potentially critical change in the patient's condition, EMR message monitor application 114 saves the information by updating PHR database 113.

The end user of EMR message monitor application 114 may be granted the ability to make changes and persist the changes to application server 120. However, in some embodiments, EMR message monitor application 114 only performs read queries (i.e., has read only access permissions) to PHR database 113. EMR message monitor application 114 may then proceed to execute a notification procedure to provide interested individuals with the ability to immediately contact the hospital. This may be initiated by transmitting this information to application server 120. An advantage of this system is that it provides interested individuals with immediate notification of critical temperature changes, which require hospital procedures to treat.

The information that is sent to application server 120 for an alert may be JavaScript Object Notation (JSON) information in the format: Body
Practitioner Name
Callback Number
Message
Urgency
Contact Information
Patient Name

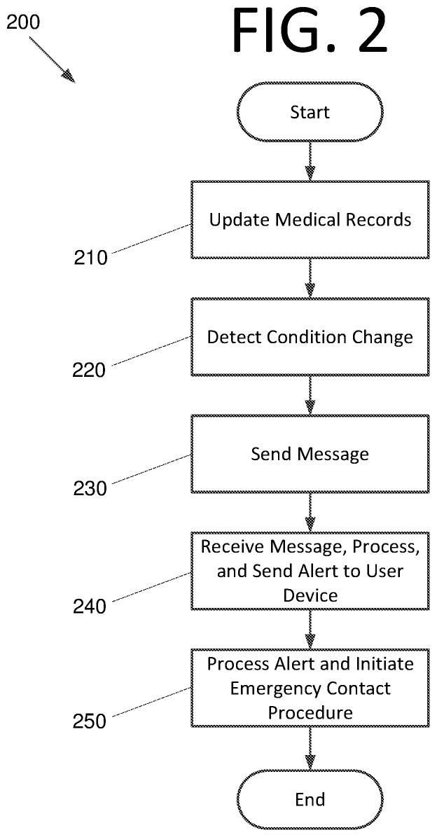

This information may be sent to application server 120 for a subsequent transaction of the communication process (see, e.g., step 230 of FIG. 2).

In some embodiments, EMR message monitor application 114 periodically scans EMR application 112 (e.g., every few minutes) searching for changes in a time stamp (e.g., an EPOCH time stamp), the patient treatment team and/or regimen (e.g., additional staff to the treatment team, staff removed from the treatment team, treatment updates or changes, etc.), changes in patient location, etc. More specifically, in some embodiments, EMR message monitor application 114 makes calls to EMR application 112 using a “fetch all” process for the desired resources that have changed by cURLing the results by a rest API call.

If a change is detected, EMR message monitor application 114 may execute the notification procedure by contacting application server 120. In some embodiments, when changes to the patient's condition are entered in EMR application 112, EMR message monitor application 114 may automatically be informed by EMR application 112 via a push notification or via a thread process that is constantly listening, and EMR message monitor application 114 may then execute the notification procedure by contacting application server 120.

Once the conditions match the algorithm assigned for the resource, a post request may then be transferred to server-side automated alert application 122 to go through middleware routing in accordance to the severity with which the resource resolution is tagged. EMR message monitor application 114 is not listening constantly in some embodiments. Rather, EMR message monitor application 114 may be periodically listening for changes at a fixed interval selected by the user (e.g., choosing an interval of 100 milliseconds to 300,000 milliseconds).

Application server 120 runs on the cloud 130 in this embodiment. In some embodiments, cloud 130 may be the IBM® Cloud. Application server 120 provides a set of cloud computing services that include infrastructure as a service (IaaS), software as a service (SaaS), and platform as a service (PaaS) offered through public, private, and/or hybrid cloud delivery models, in addition to the components that make up those clouds capabilities. Using cloud 130 may allow access to multiple client locations (e.g., institutions) simultaneously. The automated alert system may be built to current healthcare standards set by SMART on the Fast Healthcare Interoperability Resource (FHIR) API and Meaningful Use, allowing for complete integration into current hospital electronic medical records. The SMART project defines a health data layer that builds on the emerging FHIR API and resource definitions.

Server-side automated alert application 122 running on application server 120 receives data from EMR message monitor application 114 (e.g., the patient name, the type of medical condition change (e.g., a message indirectly indicating a medical condition change without violating the Health Insurance Portability and Accountability Act (HIPAA)), the contact number(s) of interested individuals, etc.), parses this data, and temporarily stores it in cache storage 124.

A user may register server-side automated alert application 122 and create a unique client ID and application ID. Once the users create their application on application server 120, the user can send his or her keys in any type of exchange to the authorities of cloud 130. Once a URL of application server 120, an authorized URL (i.e., the endpoint for application server 120 that EMR message monitor application 114 communicates with to gain authorization to database 126 to get read, write, and update permission), a name of the organization or department, the phone number associated with the client ID, and the application ID, database 126 can be populated with this information, which may make this information safe and secured through a traditional manual database row creation for a file on the client. The registration may be for documenting registered users of server-side automated alert application 122 to ensure fair use by registered licensees.

### User Device

When a critical change in condition is registered by EMR message monitor application 114, which interfaces with EMR application 110, this change is reported to server-side automated alert application 122 running on application server 120. EMR message monitor application 114 may provide information indicating the type of condition change, an indicator of the urgency of the condition, notes from a healthcare professional, contact information of authorized individuals, etc. Additionally or alternatively, the contact information of the interested individuals may be temporarily stored in database 126, cache 124, etc. for validation and transactions. Server-side automated alert application 122 then provides an alert to an authorized individual through that individual's user device 140. An application 142 running on user device 140 may receive the alert and notify the user. This may have the advantage of allowing server-side automated alert application 122 to provide additional information to the authorized individual(s) and display it in a more effective fashion. Application 142 may also allow authorized individuals to communicate with nurse workstation 110 so authorized individuals can communicate with medical staff. However, in some embodiments, application 142 may not be used and text, email, phone calls, etc. may be sent directly to user device 140.

Authorized individuals may be notified via a text (i.e., a short message service (SMS) message), a robocall, and/or email in some embodiments. The SMS message may have the number to respond back to the hospital. The phone call, if received by the authorized individual, may be placed in an outbound call immediately to the number of nurse workstation 110.

### Nurse Workstation

Nurse workstation 110 is presumably the machine location where the practitioner is authorized to make updates or signal event changes in relationship to a patient. In most hospitals, nurse workstation 110 is a desktop that runs EMR application 112 as well. In some embodiments, EMR message monitor application 114 is web-based (e.g., runs on a browser such as Microsoft Internet Explorer™ 10, Firefox™ Google Chrome™, or Safari™). In some embodiments, it should be noted that nurse workstation 110 can perform all of the functionality associated with application server 120. The user may be able to download client-side code ad run the application natively on nurse workstation 110.

FIG. 2 is a flowchart illustrating a process 200 for an automated alert system, according to an embodiment of the present invention. The process begins with authorized hospital staff accessing and updating hospital alert records at 210 to record a change in the condition of the patient. An EMR message monitor application then detects the condition change at 220 and sends a message at 230 to a server-side automated alert application running on an application server that includes information about the patient's change in condition. For instance, a PHI-sensitive payload may be sent to the application server that provides an optional message to the authorized individual's device.

The server-side automated alert application receives the message, processes it, and sends an alert to a user device at 240. The alert may be in the form of a text message, a robocall, an email, and/or a trigger to an alert application running on the user device. The alert application on the user device may then process the alert and initiate an emergency contact procedure at 250. For instance, the alert application may automatically cause the user device to dial the number of a nurse workstation. In some embodiments, the user of the user device may cancel the request for authorized individuals to come to the hospital once contact has been made.

Per the above, in some embodiments, the automated alert system may be at least partially cloud-based, allowing access to multiple client locations (e.g., institutions) simultaneously. Messages generated by the automated alert system may be sent from the cloud directly to the designated next-of-kin only when appropriate personnel are available to respond to requested call backs in some embodiments. This may be achieved by the person making the request entering a call back number that they know will have someone ready to answer the in-coming call.

If the first person on the emergency alert list does not call back within a predetermined period of time in some embodiments (e.g., five minutes), the next person on the list may be called. For instance, multiple individuals may be specified and a call order may be provided in some embodiments so that multiple individuals do not try to call the callback number at the same time. In certain embodiments, the system may call multiple individuals on the list at different periods of time. For instance, the system may call the first individual, wait ten minutes, call the second individual, wait ten minutes, call the third individual, etc. In some embodiments, the period of time may be different from one individual to the next. For instance, the period may get progressively smaller for each subsequent individual that is called, speeding up the contact process.

Once the end of the list is reached, the procedure may start again from the first person on the list in some embodiments. This procedure may repeat until contact is made. The automated alert system may also have the capabilities to continuously scan the EMR system until a change in location or a critical medical condition is detected to alert the emergency contact(s) listed in the patient record.

The EMR message monitor application may create an initial alert message record either automatically or manually responsive to input by a medical professional. The message may be encrypted to increase security and ensure HIPAA compliance. The message may be sent in a Complete Alert Message Record (CAMR) format, which is a string-padded message to send to services within the cloud (e.g., a service by Twilio®).

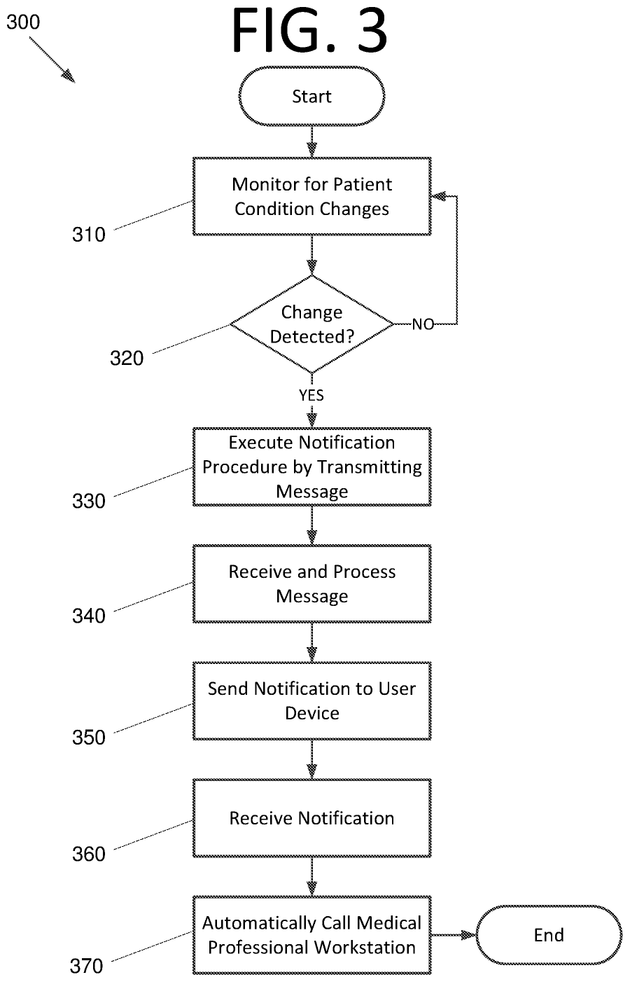

FIG. 3 is a flowchart illustrating a process 300 for an automated alert system, according to an embodiment of the present invention. The process begins with monitoring, by an EMR message monitor application, for one or more changes a condition of a patient that require notification of an authorized individual associated with a user device at 310. In some embodiments, the EMR message monitor application monitors for the one or more changes in the condition of the patient by monitoring one or more vital signs of the patient. In certain embodiments, the EMR message monitor application monitors for patient condition changes by periodically scanning the EMR application, searching for changes in a patient treatment team, a patient treatment regimen, changes in patient location, or any combination thereof. The EMR message monitor application may periodically scan the EMR application for changes by making calls to the EMR application using a “fetch all” process for desired resources that have changed. In some embodiments, when changes to the patient's condition are entered in the EMR application, the EMR application automatically informs the EMR message monitor application via a push notification or via a thread process that is constantly listening.

If one or more changes in the patient's condition are detected that require notification of the authorized individual associated with the user device at 320, the EMR application is configured to execute a notification procedure at 330 by transmitting a message including HIPAA-compliant information pertaining to the one or more changes in the condition of the patient to a server-side automated alert application. In some embodiments, the information is in JSON format and includes a practitioner name, a callback number, a message, an urgency, contact information, and a patient name. The server-side automated alert application receives and processes the message from the EMR message monitor application at 340. In some embodiments, the server-side automated alert application prioritizes notifications based on urgency. Once the message is processed, the server-side automated alert application sends a notification to the user automated alert application running on the user device at 350. The user automated alert application receives the notification at 360 and automatically places an outbound call to the medical professional workstation at 370 so the authorized individual associated with the user device can communicate with medical staff.

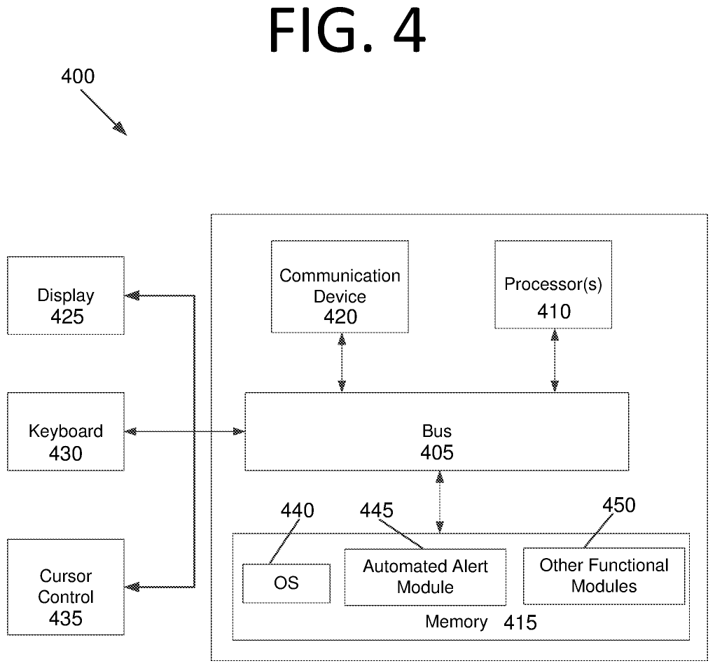

FIG. 4 is a block diagram illustrating a computing system 400 configured to provide an automated alert system, according to an embodiment of the present invention. For instance, computing system may be a server, a laptop computer, a desktop computer, a tablet, a smart phone, or any other suitable computing system without deviating from the scope of the invention. Computing system 400 includes a bus 405 or other communication mechanism for communicating information, and processor(s) 410 coupled to bus 405 for processing information. Processor(s) 410 may be any type of general or specific purpose processor, including a central processing unit (CPU), an application specific integrated circuit (ASIC), a microcontroller, or any other suitable processing hardware without deviating from the scope of the invention. Processor(s) 410 may also have multiple processing cores, and at least some of the cores may be configured to perform specific functions. Multi-parallel processing may be used in some embodiments. Computing system 400 further includes memory 415 for storing information and instructions to be executed by processor(s) 410. Memory 415 can be comprised of any combination of random access memory (RAM), read only memory (ROM), flash memory, cache, static storage such as a magnetic or optical disk, or any other types of non-transitory computer-readable media or combinations thereof. Additionally, computing system 400 includes a communication device 420, such as a transceiver and antenna, to wirelessly provide access to a communications network.

Non-transitory computer-readable media may be any available media that can be accessed by processor(s) 410 and may include volatile media, non-volatile media, removable media, and/or non-removable media.

Processor(s) 410 are further coupled via bus 405 to a display 425, such as a Liquid Crystal Display (LCD), for displaying information to a user. A keyboard 430 and/or a cursor control device 435, such as a computer mouse, are further coupled to bus 405 to enable a user to interface with computing system. However, in certain embodiments, such as those for mobile computing implementations, a physical keyboard and mouse may not be present, and the user may interact with the device solely through display 425 via a haptic interface and/or a touchpad (not shown), or through no interface at all (e.g., where a device only has an “on” button). As such, display 425, keyboard 430, and/or cursor control device 435 may not be present in some computing systems. Any type and combination of input devices may be used as a matter of design choice.

Memory 415 stores software modules that provide functionality when executed by processor(s) 410. The modules include an operating system 440 for computing system 400. The modules further include an automated alert module 445 that is configured to perform any of the automated alert operations discussed herein without deviating from the scope of the invention. Computing system 400 may include one or more additional functional modules 450 that include additional functionality.

One skilled in the art will appreciate that a “system” could be embodied as an embedded computing system, a personal computer, a server, a console, a personal digital assistant (PDA), a cell phone, a tablet computing device, or any other suitable computing device, or combination of devices. Presenting the above-described functions as being performed by a “system” is not intended to limit the scope of the present invention in any way, but is intended to provide one example of many embodiments of the present invention. Indeed, methods, systems and apparatuses disclosed herein may be implemented in localized and distributed forms consistent with computing technology, including cloud computing systems.

It should be noted that some of the system features described in this specification have been presented as modules, in order to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module. Further, modules may be stored on a computer-readable medium, which may be, for instance, a hard disk drive, flash device, RAM, tape, or any other such medium used to store data.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

The process steps performed in FIGS. 2 and 3 may be performed by a computer program, encoding instructions for the processor to perform at least the processes described in FIGS. 2 and 3, in accordance with embodiments of the present invention. The computer program may be embodied on a non-transitory computer-readable medium. The computer-readable medium may be, but is not limited to, a hard disk drive, a flash device, RAM, a tape, or any other such medium used to store data. The computer program may include encoded instructions for controlling the processor to implement the processes described in FIGS. 2 and 3, which may also be stored on the computer-readable medium.

The computer program can be implemented in hardware, software, or a hybrid implementation of both software and hardware. The computer program can be composed of modules that are in operative communication with one another, and which are designed to pass information or instructions to display. The computer program can be configured to operate on a general purpose computer, an ASIC, or any other suitable computing device.

It will be readily understood that the components of various embodiments of the present invention, as generally described and illustrated in the figures herein, may be arranged and designed in a wide variety of different configurations. Thus, the detailed description of the embodiments of the present invention, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, reference throughout this specification to “certain embodiments,” “some embodiments,” or similar language means that a particular feature, structure, or characteristic described in connection with the embodiment is included in at least one embodiment of the present invention. Thus, appearances of the phrases “in certain embodiments,” “in some embodiment,” “in other embodiments,” or similar language throughout this specification do not necessarily all refer to the same group of embodiments and the described features, structures, or characteristics may be combined in any suitable manner in one or more embodiments.

It should be noted that reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

One having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should be made to the appended claims.

What is claimed is:

1. A computer-implemented method executed in a distributed electronic healthcare data environment for monitoring of patient condition data and alert delivery, configured to improve responsiveness and reduce manual delays in communicating patient condition updates, the method comprising:
by a first electronic device having processing circuitry operably coupled to memory and a network interface,
retrieving and monitoring, via the network interface, for a change in a personal health record (PHR) repository having a plurality of patient records, each patient record having clinical data associated with at least one of a treatment team, a treatment regimen, a patient location, and a recorded observation associated with a medical condition;
selecting, based on extracted clinical data and meta attributes that are associated with the change in the PHR repository, a first programmable logic rule of a first set of programmable logic rules stored in the memory and associated with detecting a triggering condition that enables transmission of an alert notification that corresponds to the change in the PHR repository to an authorized recipient;
in response to detecting the triggering condition by applying the first programmable logic rule to the extracted clinical data and meta attributes:
generating an alert payload having information associated with the change in the PHR repository, formatted in accordance with a data interchange standard;
sending, via the network interface, to a second electronic device, an indication that includes the alert payload; and
enabling the second electronic device, based on the alert payload, to transmit the alert notification to a mobile computing device associated with the authorized recipient based on stored patient authorization data, without requiring human-in-the-loop intervention.

2. The computer-implemented method of claim 1, wherein a third electronic device is connected to the PHR repository and configured to receive messages via the network interface and send clinical messages associated with the PHR repository via the network interface, and the retrieving and monitoring step comprises:
receiving a first clinical message via the network interface from the third electronic device; and
parsing the first clinical message to extract clinical data and meta attributes that are associated with a patient record of the PHR repository.

3. The computer-implemented method of claim 2, wherein the retrieving and monitoring step further comprises:
validating that extracted clinical data of the first clinical message is compatible with extracted meta attributes of the first clinical message.

4. The computer-implemented method of claim 1, wherein the selecting step comprises:
determining that the extracted clinical data or meta attributes corresponds to at least one of a set of alphanumeric identifiers associated with patient care; and
selecting the first programmable logic rule based on the corresponding alphanumeric identifier.

5. The computer-implemented method of claim 4, wherein the set of alphanumeric identifiers is associated with Logical Observation Identifiers Names and Codes (LOINC).

6. The computer-implemented method of claim 1, wherein the first electronic device retrieves and monitors, via the network interface, for the change in the PHR repository in real time.

7. The computer-implemented method of claim 1, further comprising:
selecting, based on extracted clinical data and meta attributes that are associated with the change in the PHR repository, a second programmable logic rule of a second set of programmable logic rules stored in the memory and associated with identifying an urgency level to associate with the change;
applying the second programmable logic rule to the extracted clinical data and meta attributes to obtain the urgency level; and
wherein the alert payload includes the urgency level.

8. The computer-implemented method of claim 1, further comprising:
determining a clinical staff member to be contacted by the authorized recipient for information associated with the change in the PHR repository based on the extracted clinical data and meta attributes;
obtaining stored contact information for the clinical staff member; and
wherein the alert payload includes the stored contact information for the clinical staff member.

9. The computer-implemented method of claim 8, further comprising:
generating a transmission message that requests that the authorized recipient communicate with the clinical staff member via the stored contact information; and
wherein the alert payload includes the transmission message.

10. The computer-implemented method of claim 2, further comprising:
sending a request-to-update message over the network interface to the third electronic device requesting that the PHR repository be updated to indicate that the alert payload was sent to the second electronic device.

11. A first electronic device associated with a distributed electronic healthcare data environment for monitoring of patient condition data and alert delivery, configured to improve responsiveness and reduce manual delays in communicating patient condition updates, comprising:
a processing circuitry;
a memory;
a network interface;
wherein the memory is operationally coupled to the processing circuitry, the memory containing instructions executable by the processing circuitry whereby the processing circuitry is configured to:
retrieve and monitor, via the network interface, for a change in a personal health record (PHR) repository having a plurality of patient records, each patient record having clinical data associated with at least one of a treatment team, a treatment regimen, a patient location, and a recorded observation associated with a medical condition; and
select, based on extracted clinical data and meta attributes that are associated with the change in the PHR repository, a first programmable logic rule of a first set of programmable logic rules stored in the memory and associated with detecting a triggering condition that enables transmission of an alert notification that corresponds to the change in the PHR repository to an authorized recipient;
in response to detecting the triggering condition by applying the first programmable logic rule to the extracted clinical data and meta attributes, the processing circuitry is further configured to:
generate an alert payload having information associated with the change in the PHR repository, formatted in accordance with a data interchange standard;
send, via the network interface, over a network, to a second electronic device, an indication that includes the alert payload; and
enable the second electronic device, based on the alert payload, to transmit the alert notification to a mobile computing device associated with the authorized recipient, without requiring human-in-the-loop intervention.

12. The first electronic device of claim 11, wherein a third electronic device is connected to the PHR repository and configured to receive messages via the network interface and send clinical messages associated with updates to the PHR repository via the network interface, and the processing circuitry is further configured in the retrieving and monitoring step to:
receive a first clinical message via the network interface from the third electronic device; and
parse the first clinical message to extract clinical data and meta attributes that are associated with a patient record of the PHR repository.

13. The first electronic device of claim 12, wherein the processing circuitry is further configured in the retrieving and monitoring step to:
validate that extracted clinical data of the first clinical message is compatible with extracted meta attributes of the first clinical message.

14. The first electronic device of claim 11, wherein the processing circuitry is further configured in the retrieving step to:
determine that the extracted clinical data or meta attributes corresponds to at least one of a set of alphanumeric identifiers associated with patient care; and
select the first programmable logic rule based on the corresponding alphanumeric identifier.

15. The first electronic device of claim 14, wherein the set of alphanumeric identifiers is associated with Logical Observation Identifiers Names and Codes (LOINC).

16. The first electronic device of claim 11, wherein the processing circuitry is further configured to perform the retrieving and monitoring step in real time.

17. The first electronic device of claim 11, wherein the processing circuitry is further configured to:
select, based on extracted clinical data and meta attributes that are associated with the change in the PHR repository, a second programmable logic rule of a second set of programmable logic rules stored in the memory and associated with identifying an urgency level to associate with the change;
apply the second programmable logic rule to the extracted clinical data and meta attributes to obtain the urgency level; and
wherein the alert payload includes the urgency level.

18. The first electronic device of claim 11, wherein the processing circuitry is further configured to:
determine a clinical staff member to be contacted by the authorized recipient for information associated with the change in the PHR repository based on the extracted clinical data and meta attributes;
obtain stored contact information for the clinical staff member;
generate a transmission message that requests that the authorized recipient communicate with the clinical staff member via the stored contact information; and
wherein the alert payload includes the stored contact information for the clinical staff member and the transmission message.

19. The first electronic device of claim 12, wherein the processing circuitry is further configured to:
send a request-to-update message over the network interface to the third electronic device requesting that the PHR repository be updated to indicate that the alert payload was sent to the second electronic device.

20. A computer-implemented system in a distributed electronic healthcare data environment for monitoring of patient condition data and alert delivery, configured to improve responsiveness and reduce manual delays in communicating patient condition updates, comprising:
a medical facility computing infrastructure including:
at least one workstation device having a processor, memory, and network interface;
at least one server system comprising a processor, memory, and network interface; and
a software application platform comprising a plurality of integrated modules executable across the workstation device and server system;
the software application platform including an EMR monitoring module executable on the workstation device and a server-side automated alert module executable on the server system, with the EMR monitoring module configured to:
retrieve and monitor, via the network interface of the workstation device, for a change in a personal health record (PHR) repository having a plurality of patient records, each patient record having clinical data associated with at least one of a treatment team, a treatment regimen, a patient location, and a recorded observation associated with a medical condition;
select, based on extracted clinical data and meta attributes that are associated with the change in the PHR repository, a first programmable logic rule of a first set of programmable logic rules stored in the memory and associated with detecting a triggering condition that enables transmission of an alert notification that corresponds to the change in the PHR repository to an authorized recipient; and
in response to detecting the triggering condition by applying the first programmable logic rule to the extracted clinical data and meta attributes, the EMR monitoring module is further configured to:
generate an alert payload having information associated with the change in the PHR repository, formatted in accordance with a data interchange standard;
send, via the network interface, to the server system, an indication that includes the alert payload; and
enable the server-side automated alert module, based on the alert payload, to transmit the alert notification to a mobile computing device associated with the authorized recipient based on stored patient authorization data, without requiring human-in-the-loop intervention.
